(19) United States
(12) Plant Patent Application Publication
(10) Pub. No.: US 2018/0042154 P1
(43) Pub. Date: Feb. 8, 2018

(54) PLUM TREE NAMED 'POLARIS'

(50) ;

(71) Applicant: STARGROW CULTIVAR DEVELOPMENT (PROPRIETARY) LIMITED, Stellenbosch (ZA)

(72) Inventor: Jeanne Fourie, Stellenbosch (ZA)

(21) Appl. No.: 15/330,097

(22) Filed: Aug. 5, 2016

(51) Int. Cl. A01H 5/08

(52) U.S. Cl. PLT/184

(57) ABSTRACT

‘Polaris’ is new plum tree variety that can be distinguished from other plum tree varieties by a combination of characteristics that include harvesting date, flesh color, fruit shape, and eating quality.

Genus and species: The plum tree variety of this invention is botanically identified as Prunus salicina.

Variety denomination: The variety denomination is ‘Polaris’.

### BACKGROUND OF THE INVENTION

The invention refers to a new plant variety of plum tree (Prunus salicina) named ‘Polaris’. The new Prunus variety is product of a planned and controlled breeding program for new Prunus varieties. The objective of the breeding program is to select new high quality Prunus varieties with good production and outstanding eating quality

The new Prunus variety was obtained through a controlled open pollinated cross. Seedlings of the population was planted on own root in 1999. The first fruit of ‘Polaris’ was observed in 2003 from the seedling trees. Buds from seedling was selected from the top-grafted trees and 5 trees was propagated on Marianna rootstock as second generation trees in 2005. Fruit form the second generation trees was first observed in 2008. ‘Polaris’ was selected in 2009 after the second fruit crop form the second generation trees for further development because of the unique fruit quality traits observed over the two seasons consecutively.

### BRIEF SUMMARY OF THE INVENTION

The following traits have been repeatedly observed and are determined to be unique characteristics of ‘Polaris’ which in combination distinguish this plum tree as a new and distinct variety:
1. Harvesting date;
2. Flesh color;
3. Fruit shape; and
4. Eating quality.

The fruit of ‘Polaris’ ripens between “Fortune” and “Flavor King”. Compared to “Fortune”, fruit from ‘Polaris’ ripens 10 days after “Fortune”. Compared to “Flavor King” fruit of ‘Polaris’ develops a yellow flesh color with some red bleeding distinctly from the skin while fruit of “Flavor King” has pinkish red flesh. Fruit of ‘Polaris’ has a truncate to depressed apex where the apex of “Flavor King” fruit is rounded.

Fruit of ‘Polaris’ has unique eating quality. The orange-pink flesh is firm non-melting with a “rose-water” type flavor and a high sugar content.

### BRIEF DESCRIPTION OF THE PHOTOGRAPHS

The accompanying photographs illustrate the appearance of the new apple tree in production, fruit and internal quality of the fruit. The color of the photographs might vary slightly compared to the variety description, however the botanical description are the most accurately description of the true color of this new variety.

FIG. 1. A central axis cut of fruit illustrating the yellow flesh with bleeding in the flesh.

FIG. 2. A fully developed fruit, close up, on a tree grown in an orchard, illustrating colour development and shape of the fruit.

FIG. 3. A fully developed fruit, close-up, on a tree grown in an orchard illustrating the colour development and shape of this new variety.

FIG. 4. Fully developed leaf illustrating the color, shape of the leaf and margin incisions.

FIG. 5. Illustrates a three year old growing tree on a trellis system of this new variety in an orchard.

FIG. 6. Illustrates a bearing tree in a natural growing state.

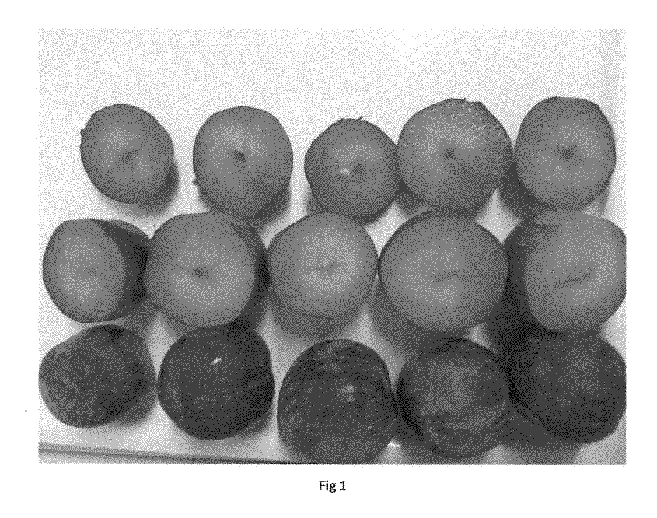

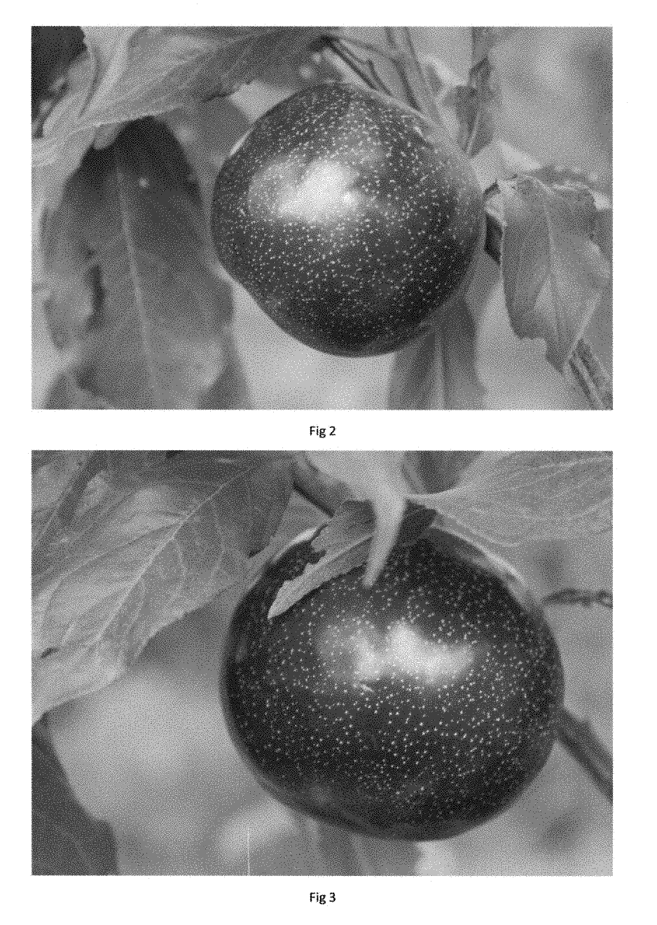

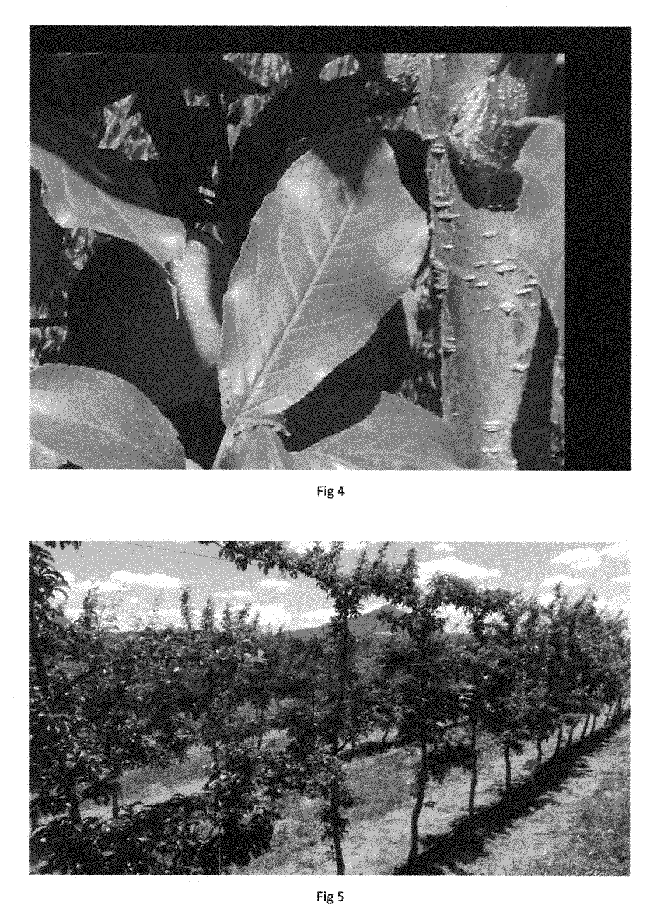

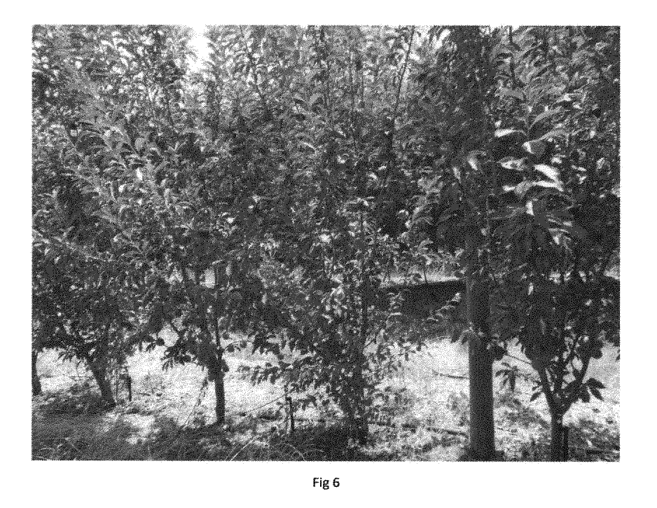

### DETAILED BOTANICAL DESCRIPTION

The new Prunus variety ‘Polaris’ has not been observed under all possible environmental conditions. The phenotype of the new variety may vary with variations in environmental conditions such as temperature, light intensity and day length without any change in the genotype of the tree.

The photographs and description is taken from trees grown at the Martinusrust Farm situated near Clanwilliam in the Western Cape, South Africa. General commercial orchard practice is applied to the trees. The climate is temperate continental with low temperatures during winter and high temperatures during summer. The soil type is sandy with a double drip irrigation system.

The detailed botanical description is taken from over several fruiting season of trees budded on a standard plum rootstock. The trees were four years old on a trellis system when the first observations were made. Measurements and numerical values represents averages for typical plants and plant parts. The actual measurements of any individual plant of plant parts, or any group of plants of plant parts, may vary from the stated average.
Plant description:
General:
   Size.—Medium.
   Vigor.—Medium.
   Productivity.—Productive/good.
   Form.—Pyramid.
   Bearer.—Regular bearer.
   Disease resistance/susceptibility.—No particular plant/fruit resistance/susceptibility has been observed.
   Insect resistance/susceptibility.—No particular plant/fruit resistance/susceptibility has been observed.
   Trunk circumference.—Approximately 17 cm, measured +−40 cm above ground.
   Trunk color.—Grey-brown.
   Branch length.—Approximately 206 cm (2 branches), 116 cm (1 branch).
   Branch size.—Diameter approximately 7 cm (measured +−30 cm from trunk).
   Branch texture.—Medium.
   Branch color.—Reddish-brown.
   Lenticels.—Density: Count, approximately 21 lenticels within a 2 cm² area on the trunk Density: Count, approximately 31 lenticels within a 2 cm² area on the branch Color: Light Brown on both trunk and branch Size: Trunk lenticels approximately 5 mm²; Branch lenticels approximately 0.9 mm² Length: Trunk approximately 5 mm; Branch approximately 1.63 mm Width: Trunk approximately 1 mm; Branch approximately 0.57 mm Number of lenticels: Present, medium.
Foliage description:
Leaves:
   Form.—Elliptic.
   Leaf blade (ration of length to width).—Elongated.
   Angle at apex.—Acute.
   Leaf blade tip.—Slightly curved downward.
Fruit description:
General:
   1st pick 18 Jan, 2nd pick 25 Jan.
   Maturity when described.—Firm, 6-8 kg with 11 mm penetrometer probe.
   Season ripening.—Mid-season.
Position of maximum diameter: Middle of fruit.
Symmetry about the suture: Symmetric.
Shape of base: Depressed.
Size: Diameter perpendicular to suture plane — Approximately 55-65 mm.
Average weight: Approximately 110 g.
Form:
   Viewed from apex.—Round, circular, symmetrical.
   Viewed from side, facing suture.—Symmetric, rounded, truncate-to-depressed apex.
   Viewed from side, perpendicular to suture.—Rounded, truncate-to-depressed apex, symmetrical.
   Apex shape.—Truncate/flattened.
   Apex base.—Depressed.
Fruit stem cavity:
   Shape.—Flaring.
   Depth.—Medium.
   Width.—Broad.
Fruit stem:
   Color.—Medium green.
   Adherence to stone.—Medium to weak.
Fruit skin:
   Thickness.—Medium.
   Adherence to flesh.—Medium.
   Surface texture.—Smooth.
   Pubescence.—Absent.
   Ground color.—Yellow-green.
   Overcolor.—Red-purple.
   Relative area of overcolor.—Large.
   Pattern of overcolor.—Solid flush.
   Roughness.—Absent.
   Tenacity.—Firm to flesh.
   Tendency to crack.—Absent.
Flesh:
   Ripens.—Evenly.
   Texture.—Crisp-juicy.
   Fibers.—Medium.
   Flavor.—Neutral, sweet.
   Brix.—Approximately 18-20°.
   Juice.—Medium to abundant.
   Aroma.—Slight.
   Color.—Yellow with bleeding.
   Anthocyanin color around stone.—Absent.
   Anthocyanin color of flesh.—Medium.
   Acidity.—Low to Medium — 2.35 g/100 ml.
   Sugar content.—High, approximately 18-20° Brix.
   Eating quality.—Very good.
   Firmness.—Firm to crisp texture.
   Fruit shipping and keeping quality.—Good, for four to eight weeks.
Stone:
   Stone freeness.—Cling.
   Degree of adherence to flesh.—Medium.
   Stone size.—Medium.
   Stone form.—Viewed from side: Narrow elliptic Viewed from ventral end.: Narrow elliptic.
   Stone shape.—Flattened symmetrical.
   Stone tendency to split.—No tendency observed.
   Suture line.—Medium.

What is claimed is:

1. A new and distinct variety of plum tree having the characteristics substantially as described and illustrated herein.
